(19) United States
(12) Patent Application Publication
(10) Pub. No.: US 2005/0148268 A1
(43) Pub. Date: Jul. 7, 2005

(54) Non-woven composite fabric and product made therefrom

(75) Inventor: Jung Chi Tai, Tainan (TW)

Correspondence Address: THOMAS, KAYDEN, HORSTEMEYER & RISLEY, LLP, 100 GALLERIA PARKWAY, NW, STE 1750, ATLANTA, GA, 30339-5948, US

(73) Assignee: Kang Na Hsiung Enterprise Co., Ltd.

(21) Appl. No.: 11/030,857

(22) Filed: Jan. 7, 2005

(30) Foreign Application Priority Data
Jan. 7, 2004 (TW) 93100320

(51) Int. Cl. B32B 5/26; D04H 1/00; B32B 27/12; D04H 3/00

(52) U.S. Cl. 442/414; 442/327; 442/361; 442/364; 442/381; 442/400; 442/401; 442/394

(57) ABSTRACT

A non-woven composite fabric, comprising at least two layers from a carded web, spunbonded web, meltblown web, and air-laid web. At least one layer of the above webs includes an adhesive fiber, and the adhesive fiber includes a maleic acid or maleic anhydride grafted olefin polymer.

### BACKGROUND OF THE INVENTION

1. Field of the Invention

The present invention relates to a non-woven composite fabric, and more particularly to a non-woven composite fabric having high peeling strength, made from an adhesive fiber containing maleic acid or maleic anhydride grafted olefin polymers, and products made from the non-woven composite fabric.

2. Description of the Related Art

Non-woven fabrics have been extensively used in household and medical applications, for example, used as panty liners, disposable diapers, sanitary napkins, and surgical clothes. Generally, a non-woven fabric is formed by consolidating multiple layers of different webs. For example, in U.S. Pat. No. 4,196,245, it is disclosed that a meltblown web is composited with other webs such as spunbonded web, wet-laid web, carded web, and air-laid web.

The conventional non-woven composite fabric, however, when made into sanitary products (such as panty liners) suffers from insufficient peeling strength. Thus, webs peel off easily.

U.S. Pat. No. 4,950,541 and U.S. Pat. No. 5,372,885 disclose a bi-component adhesive fiber, including a core made of polyester and polyamide and a sheath made of polyethylene having succinic acid grafted thereon.

U.S. Pat. No. 5,185,199 discloses a fabric containing a bi-component adhesive fiber. The adhesive fiber includes linear low density polyethylene (LLDPE) and high density polyethylene (HDPE) grafted with maleic acid to obtain polyethylene having succinic acid grafted thereon.

EP 0 366 379 first discloses a core/sheath type adhesive fiber similar to the present invention, including a polyester core and a sheath of ethylene/vinyl acetate (EVA) copolymer. EP 0 366 379 discloses that such adhesive fiber has good adhesion to other fiber (such as polyester fiber) and thus can be blended with other fiber to obtain a bonded article.

The above conventional patents, however, do not disclose that an adhesive fiber can be used to produce non-woven composite fabric and even a non-woven composite fabric product (such as diaper).

### SUMMARY OF THE INVENTION

An object of the present invention is to provide a non-woven composite fabric with excellent adhesion between each web for preventing peeling off. The non-woven composite fabric of the present invention has good peeling strength, mechanical direction strength, and cross direction strength. The non-woven composite fabric of the present invention can be a wet wipes, clothing interliner, loop of a hook-and-loop fastner, filtermedia, or shoe material.

Another object of the present invention is to provide a product made from the above non-woven composite fabric, with excellent adhesion between each web for preventing peeling off. The product made from the non-woven composite fabric can be a diaper, sanitary napkin, or panty liner.

To achieve the above objects, the adhesive fiber-containing non-woven composite fabric of the present invention includes at least two layers from a carded web, spunbonded web, meltblown web, and air-laid web. At least one layer of the above webs includes an adhesive fiber. The adhesive fiber includes maleic acid or maleic anhydride grafted olefin polymers.

The non-woven composite fabric of the present invention can be further employed to produce various non-woven composite fabric products, such as diapers, sanitary napkins, or panty liners.

### DETAILED DESCRIPTION OF THE INVENTION

The non-woven composite fabric of the present invention includes at least two layers from a carded web, spunbonded web, meltblown web, and air-laid web. The two layers can be two webs of the same type or two webs of different types. For example, the non-woven composite fabric of the present invention can include at least two carded webs. The webs can be bonded by at least one method of hot air through, needle punch, or water jet. After the webs are bonded, the webs can be further processed by film laminating or extrusion coating to form a composite non-woven fabric.

The feature of the present invention is the non-woven composite fabric comprising an adhesive fiber to improve its peeling strength among webs. The adhesive fiber includes maleic acid or maleic anhydride grafted olefin polymers.

The adhesive fiber used in the present invention can be a mono-component fiber or a bi-component fiber. When the adhesive fiber is a bi-component fiber, the bi-component fiber can be sheath and core type or side by side type.

When the adhesive fiber is sheath and core type, the sheath layer of the adhesive fiber can be a maleic acid or maleic anhydride grafted olefin polymer. The core layer of the adhesive fiber is not limited and can be polyethylene, polypropylene, polyester, or polyamide.

The present invention uses a special adhesive fiber to produce non-woven composite fabric. Since the adhesive fiber contains a hydrophilic group, that is, carboxy (—COOH) or carboxylate (—COO^{—}), it has good affinity with other materials. Therefore, in the non-woven composite fabric of the present invention, each web has good adhesion to same and other fiber and does not peel off easily. Therefore, the non-woven composite fabric of the present invention has good peeling strength, mechanical direction strength, and cross direction strength.

According to the present invention, suitable fibers and webs can be chosen to enable the adhesive fiber-containing non-woven composite fabric to function as a wetwipes, clothing interliner, loop of a hook-and-loop fastner, filter media, or shoe material. A wet wipes can be obtained by immersing the non-woven composite fabric of the present invention in water or suitable liquid. When the clothing interliner of the present invention is heated (such as ironed by an iron), it can be conveniently bonded to the cloth due to the adhesive fiber contained therein, which is very convenient. In the loop of the hook-and-loop fastner of the present invention, fibers will have good adhesion therebetween due to the presence of the adhesive fiber. Thus, the lifetime of the hook-and-loop fastner will be extended.

The non-woven composite fabric of the present invention can be further processed to produce various products, such as diapers, sanitary napkins, or panty liners. For example, a water-impermeable film (such as PE film) can be laminated to the bottom of the non-woven composite fabric of the present invention, and embossed, and a leakage preventing sidewall is added, thus producing a non-woven composite fabric product such as a diaper, sanitary napkin, or panty liner. The non-woven composite fabric product of the present invention also has good peeling strength, mechanical direction strength, and cross direction strength.

The following examples are intended to illustrate the process and the advantages of the present invention more fully without limiting its scope, since numerous modifications and variations will be apparent to those skilled in the art.

### COMPARATIVE EXAMPLE 1

### Non-Woven Composite Fabric for Panty Liner

The composition of the non-woven composite fabric of this comparative example is shown in Table 1 and physical properties are shown in Table 3. PP/PE core/sheath composite fiber indicates that the core portion is polypropylene (PP) and the sheath portion is polyethylene (PE).

TABLE 1

| Layer | Fiber | Web |
|---|---|---|
| First | PP/PE core/sheath | Carded web |
| | composite fiber (2 d × 51 mm) | (8 gsm) |
| Second | PP/PE core/sheath | Carded web |
| | composite fiber (2 d × 6 mm) | (29.5 gsm) |
| | Pulp | Air-laid web |
| | | (56.5 gsm) |
| Third | PP/PE core/sheath | Carded web |
| | composite fiber (2 d × 51 mm) | (7 gsm) |
| Fourth | PP/PE core/sheath | Carded web |
| | composite fiber (2 d × 51 mm) | (7 gsm) |

gsm is the unit of basis weight
gsm = grams per square meters = g/m²

### EXAMPLE 1

### Non-Woven Composite Fabric for Panty Liner

The composition of the non-woven composite fabric of this example is shown in Table 2 and physical properties are shown in Table 3. PP/PE core/sheath composite fiber indicates that the core portion is polypropylene (PP) and the sheath portion is polyethylene (PE). PET/MPE core/sheath composite fiber indicates that the core portion is polyester and the sheath portion is modified polyethylene (MPE), which is a mixture of polyethylene and maleic anhydride grafted polyethylene.

TABLE 2

| Layer | Fiber | Web |
|---|---|---|
| First | PP/PE core/sheath | Carded web |
| | composite fiber (2 d × 51 mm) | (7 gsm) |
| Second | PET/MPE core/sheath | Air-laid web |
| | composite fiber (1.5 d × 3 mm) | (11 gsm) |
| | Pulp | Air-laid web |
| | | (44 gsm) |
| Third | PET/MPE core/sheath | Carded web |
| | composite fiber (1.5 d × 38 mm) | (1.8 gsm) |
| | PP/PE core/sheath | Carded web |
| | composite fiber (2 d × 6 mm) | (7.2 gsm) |
| Fourth | PET/MPE core/sheath | Carded web |
| | composite fiber (1.5 d × 38 mm) | (1.8 gsm) |
| | PP/PE core/sheath | Carded web |
| | composite fiber (2 d × 51 mm) | (7.2 gsm) |

TABLE 3

| | Comparative | |
|---|---|---|
| Physical property | Example 1 | Example 1 |
| Basis weight (g/m²) | 105.3 | 84.0 |
| Peeling strength (g/25 mm) | 30.5 | 49.2 |
| Thickness (mm) | 1.31 | 2.06 |
| Density (g/cm³) | 0.0804 | 0.0408 |

It can be seen from Table 3 that the non-woven composite fabric for panty liner obtained from Example 1, using adhesive fiber (PET/MPE core/sheath composite fiber), has a higher peeling strength than that without using adhesive fiber.

### COMPARATIVE EXAMPLE 2

### Active Carbon Non-Woven Composite Fabric

The composition of the active carbon non-woven composite fabric of this comparative example is shown in Table 4 and physical properties are shown in Table 6.

TABLE 4

| Layer | Fiber | Web |
|---|---|---|
| First | PP/PE core/sheath | Carded web |
| | composite fiber | (20 gsm) |
| Second | PP/PE core/sheath | Air-laid web |
| | composite fiber | (20 gsm) |
| | Active carbon particles | Air-laid web |
| | | (65 gsm) |
| Third | PP/PE core/sheath | Carded web |
| | composite fiber | (20 gsm) |

### EXAMPLE 2

### Active Carbon Non-Woven Composite Fabric

The composition of the active carbon non-woven composite fabric of this example is shown in Table 5 and physical properties are shown in Table 6.

TABLE 5

| Layer | Fiber | Web |
|---|---|---|
| First | PP/PE core/sheath | Carded web |
| | composite fiber | (20 gsm) |
| Second | PET/MPE core/sheath | Air-laid |
| | composite fiber | (20 gsm) |
| | Active carbon particles | Air-laid web |
| | | (65 gsm) |
| Third | PP/PE core/sheath | Carded web |
| | composite fiber | (20 gsm) |

TABLE 6

| | Comparative | |
|---|---|---|
| Physical property | Example 2 | Example 2 |
| Basis weight (gsm) | 125 | 125 |
| MD strength (kg/25 mm) | 3.6 | 5.1 |

MD strength = mechanical direction strength

It can be seen from Table 6 that the active carbon non-woven composite fabric obtained from Example 2, using adhesive fiber (PET/MPE core/sheath composite fiber), has a higher MD strength than that without using adhesive fiber.

### COMPARATIVE EXAMPLE 3

### Wet cloth

The wet cloth of this example is a carded web made of 65% Rayon fiber mixed with 35% PP/PE core/sheath composite fiber. Physical properties are shown in Table 8.

### EXAMPLE 3

### Wet Wipes

The composition of the wet wipes of this example is shown in Table 7 and physical properties are shown in Table 8.

TABLE 7

| Layer | Fiber | Web |
|---|---|---|
| First | PP/PE core/sheath | Carded web |
| | composite fiber | (8 gsm) |
| Second | 60% Rayon fiber | Carded web |
| | 40% PET/MPE core/sheath | (16 gsm) |
| | composite fiber | |
| Third | PP/PE core/sheath | Carded web |
| | composite fiber | (4 gsm) |

TABLE 8

| | Comparative | |
|---|---|---|
| Physical property | Example 3 | Example 3 |
| Basis weight (gsm) | 28.8 | 30.0 |
| MD strength (kg/25 mm) | 2.4 | 2.7 |
| CD strength (kg/25 mm) | 0.29 | 0.55 |

MD strength = mechanical direction strength
CD strength = cross direction strength

It can be seen from Table 8 that the wet wipes obtained from Example 3, using adhesive fiber (PET/MPE core/sheath composite fiber), has a higher MD strength and CD strength than that without using adhesive fiber.

The foregoing description of the preferred embodiments of this invention has been presented for purposes of illustration and description. Obvious modifications or variations are possible in light of the above teaching. The embodiments chosen and described provide an excellent illustration of the principles of this invention and its practical application to thereby enable those skilled in the art to utilize the invention in various embodiments and with various modifications as are suited to the particular use contemplated. All such modifications and variations are within the scope of the present invention as determined by the appended claims when interpreted in accordance with the breadth to which they are fairly, legally, and equitably entitled.

1. A non-woven composite fabric, comprising at least two layers from a carded web, spunbonded web, meltblown web, and air-laid web,
wherein at least one layer of the above webs includes
an adhesive fiber comprising maleic acid or
maleic anhydride grafted olefin polymers.

2. The non-woven composite fabric as claimed in claim 1, wherein the adhesive fiber is a mono-component fiber.

3. The non-woven composite fabric as claimed in claim 1, wherein the adhesive fiber is a bi-component fiber.

4. The non-woven composite fabric as claimed in claim 3, wherein the adhesive fiber is sheath and core type, and includes a core layer and a sheath layer.

5. The non-woven composite fabric as claimed in claim 4, wherein the sheath layer of the adhesive fiber is a mixture of polyethylene and maleic acid or maleic anhydride grafted olefin polymer.

6. The non-woven composite fabric as claimed in claim 1, wherein the webs are bonded by at least one method of hot air through, needle punch, or water jet.

7. The non-woven composite fabric as claimed in claim 6, wherein after the webs are bonded, the webs are further processed by film laminating or extrusion coating to form a composite non-woven fabric.

8. The non-woven composite fabric as claimed in claim 1, comprising at least one carded web and at least one air-laid web.

9. The non-woven composite fabric as claimed in claim 1, wherein the non-woven composite fabric can be used to form a wet wipes, clothing interliner, loop of a hook-and-loop fastner, filter media, or shoe material.

10. The non-woven composite fabric as claimed in claim 1, comprising an air-laid web of active carbon particles.

11. The non-woven composite fabric as claimed in claim 10, wherein the non-woven composite fabric can be used to form an active carbon filter.

12. A product of an adhesive fiber-containing non-woven composite fabric, comprising a non-woven composite fabric, wherein the non-woven composite fabric includes at least two layers from a carded web, spunbonded web, meltblown web, and air-laid web,
wherein at least one layer of the above webs includes
an adhesive fiber, wherein the adhesive fiber includes a maleic acid or maleic anhydride grafted olefin polymer.

13. The product as claimed in claim 12, which is a diaper, sanitary napkin, or panty liner.
